(19) United States
(12) Patent Application Publication
(10) Pub. No.: US 2007/0255133 A1
(43) Pub. Date: Nov. 1, 2007

(54) Method for vessel segmentation in tomographic volume data, tomography system and storage medium

(76) Inventor: Michael Suhling, Erlangen (DE)

Correspondence Address: HARNESS, DICKEY & PIERCE, P.L.C., P.O.BOX 8910, RESTON, VA, 20195, US

(21) Appl. No.: 11/790,708

(22) Filed: Apr. 27, 2007

(30) Foreign Application Priority Data
Apr. 28, 2006 (DE) 10 2006 019 918.9

(51) Int. Cl. A61B 5/05

(52) U.S. Cl. 600/425

(57) ABSTRACT

A method is disclosed for mathematically generating a virtual catheter in tomographic volume data in which the virtual catheter is assembled from a multiplicity of catheter segments, each of which being separately describable in a closed mathematical expression and each being approximated to the measured volume data by error calculation and variation of their parameters. In at least one embodiment, transition criteria between the surfaces and the center lines of the virtual segments which create smooth transitions, and thus already predetermine a multiplicity of parameters of the segment following in each case, are used in order to reduce the parameters to be adapted. The description of the envelope of the segments and of the center line of the segments by way of second-degree polynomials is particularly suitable for representation in a mathematically closed form. In addition, in another embodiment, a tomography system is disclosed for carrying out an embodiment of the method and a storage medium with program code is disclosed, for an embodiment of the method.

### PRIORITY STATEMENT

The present application hereby claims priority under 35 U.S.C. §119 on German patent application number DE 10 2006 019 918.9 filed Apr. 28, 2006, the entire contents of which is hereby incorporated herein by reference.

### FIELD

Embodiments of the invention generally relate to a method for the mathematical generation of a virtual catheter in tomographic volume data. For example, they may relate to one in which the virtual catheter is assembled from a multiplicity of catheter segments which can be described in a particularly simple manner and which are in each case approximated to the measured volume data by error calculation and variation of their parameters. In addition, embodiments of the invention also generally relate to a tomography system for carrying out the method and/or a storage medium with program code for the method according to an embodiment of the invention.

### BACKGROUND

Various techniques for vessel segmentation in tomographic volume data are known in the prior art. By way of example, reference is made to the following documents:

[1] O. Wink, W. Niessen, and M. Viergever, “Fast Delineation and Visualization of Vessels in 3-D Angiographic Images”, IEEE Trans. Med. Imag., Vol. 19, No. 4, pages 337-346, April 2000; [2] A. Frangi, W. Niessen, R. Hoogeveen, et al., “Model-based Quantitation of 3-D Magnetic Resonance Angiographic Images”, IEEE Trans. Med. Imag., Vol. 18, No. 10, pages 946-956, October 1999; and Reference [3] to S. Worz and K. Rohr, “A new 3D parametric intensity model for accurate segmentation and quantification of human vessels”, in Proceedings of the Fifth International Conference on Medical Image Computing and Computer Assisted Intervention (MICCAI'04), ser. Lecture Notes in Computer Science, Vol. 3216. Saint-Malo, France: Springer-Verlag Berlin Heidelberg, Sep. 26-30, 2004, pages 491-499.

In document [1], the center line of the vessel is approximated by a multiplicity of straight lines joined together. The center lines and the vessel surface are determined iteratively by adapting rays in a plane normal to the running straight center line. The problem in this method is that when the vessel boundaries are not clearly defined, they are easily penetrated and the method proceeds into emptiness.

In document [2], it is proposed to approximate the center line of a vessel by way of an “active contour approach”, wherein a multiplicity of markings must first be set by the operator for this purpose. Once the center line has been adapted, the contour is fitted by an “active surface model”. Since the center line and the vessel lumen are determined in two separate independent steps, the determination of the center line, in particular, is limited in its accuracy. In addition, the user interaction for determining the center line is very high. The precision of adaptation is correspondingly limited.

In document [3], finally, a method is proposed in which a multiplicity of cylinders individually approximated to the vessel lumen are used and joined together. These cylinders have no functional relations to one another and can also replicate curvatures only inadequately.

### SUMMARY

In at least one embodiment, the invention provides a method for vessel segmentation in tomographic volume data which carries out the segmentation within a short computing time, on the one hand, and with optimum adaptation to the actual contour of the vessels, on the other hand. In at least one embodiment, the invention represents a tomography system for carrying out this method and a storage medium with program code for the method according to at least one embodiment of the invention.

The inventor has found that at least one embodiment of a method for the mathematical generation of a virtual catheter in tomographic volume data is particularly advantageous if the catheter to be generated virtually is composed of a multiplicity of catheter segments each of which can be described separately in a closed mathematical expression and which are in each case approximated to the measured volume data by error calculation and variation of their parameters. In this context, both the center lines and the surfaces of the catheter segments generated should merge into one another.

It is particularly advantageous to use for the reduction of the parameters to be adapted transition criteria between the surfaces and the center lines of the virtual segments which generate smooth transitions. A multiplicity of parameters of the in each case following segment, which should otherwise have to be recalculated for each virtual catheter segment, can thus already be predetermined. The description of the envelope of the segments and of the center line of the segments by second-degree polynomials is particularly suitable for the representation in a mathematically closed form.

Accordingly, in at least one embodiment, the inventor proposes a method for vessel segmentation in tomographic volume data, preferably in CT volume data, comprising the following method steps:
tomographic volume data of a patient are reconstructed, the vessel representations having image values which significantly differ from the environment,
a picture element in the volume examined (voxel), marked by an operator in a tomographic representation, is received as starting point in a vessel of interest,
around this starting point, adjacent voxels are automatically marked, the image values of which are in the vicinity of the originally marked voxel within predetermined image value limits until the sum of all marked voxels reveals a preferred spatial orientation and a first radius perpendicularly to the preferred spatial orientation,
beginning at least in one direction of the preferred spatial orientation, a multiplicity of virtual catheter segments are joined together which have a center line and a circular envelope with one radius or an ellipsoidal envelope with two semi-axes and meet the following conditions:
each component of the center line of a catheter segment is described by a second-degree polynomial,
the radius of the envelope of each catheter segment is described by a second-degree polynomial,
the center lines of adjacent catheter segments merge into one another,
the envelopes of adjacent catheter segments merge into one another, wherein
for each continuing catheter segment, the parameters of the describing polynomials are varied until the course of the virtual catheter is optimally adapted to the course of the vessel.

In this context, it may be advantageous if the continuing catheter segments continuously merge into one another at least with respect to their center lines and the center lines of the continuing catheter segments exhibit the same spatial direction at their contact points.

This correspondingly also applies to the envelopes of adjacent catheter segments which advantageously should continuously merge into one another or, in an improved variant, are selected in such a manner that the surface tangents of the envelopes of adjacent catheter segments continuously merge into one another.

These abovementioned criteria ensure not only a natural course of the catheter created virtually but additionally reduce the degrees of freedom of the in each case following catheter segment and thus also the number of freely determinable parameters so that a drastic shortening of the computing time is also advantageously obtained.

For example, the center lines of the catheter segments S⁽ⁿ⁾ can be described by the following formula in the Cartesian system of coordinates:

$${\vec{c}}^{( n )} ( t ) = ( \begin{matrix}{x}^{( n )} ( t ) \\ {y}^{( n )} ( t ) \\ {z}^{( n )} ( t )\end{matrix} ) = ( \begin{matrix}\frac{1}{2} {a}_{12}^{( n )} {t}^{2} + {a}_{11}^{( n )} t + {a}_{10}^{( n )} \\ \frac{1}{2} {a}_{22}^{( n )} {t}^{2} + {a}_{21}^{( n )} t + {a}_{20}^{( n )} \\ \frac{1}{2} {a}_{32}^{( n )} {t}^{2} + {a}_{31}^{( n )} t + {a}_{30}^{( n )}\end{matrix} ) ,$$

where n is the index for the consecutive number of the catheter segment, x, y, z are the Cartesian coordinates, t is an arbitrary parameter which increases monotonously with the length of the center line and aᵢⱼ are the parameters to be determined in the polynomials.

The radius of the center line of the catheter segments can also be described by the following formula in the Cartesian system of coordinates:

$${r}^{( n )} ( t ) = \frac{1}{2} {b}_{2}^{( n )} {t}^{2} + {b}_{1}^{( n )} t + {b}_{0}^{( n )} ,$$

where n is the consecutive number of the catheter segment, x, y, z are the Cartesian coordinates, t is an arbitrary parameter which increases monotonously with the length of the center line and bᵢ are the parameters to be determined in the polynomials.

According to at least one embodiment of the invention, the mathematical effort is also reduced if, after the first catheter segment is known, the following applies to the parameters of the center line of the (n+1)th catheter segment mentioned thereafter:

$$\begin{matrix}{a}_{10}^{( n + 1 )} = {x}^{( n )} ( {T}_{n} ) \\ {a}_{20}^{( n + 1 )} = {y}^{( n )} ( {T}_{n} ) \\ {a}_{30}^{( n + 1 )} = {z}^{( n )} ( {T}_{n} )\end{matrix} \} ,$$

where Tₙ is the last parameter t and x⁽ⁿ⁾(Tₙ), y⁽ⁿ⁾(Tₙ), z⁽ⁿ⁾(Tₙ) are the last coordinates of the center line of the nth catheter segment.

After the first catheter segment is known, the following can similarly apply to the parameters of the radius of the envelope of the (n+1)th catheter segment mentioned thereafter:
b₀⁽ⁿ⁺¹⁾=r⁽ⁿ⁾(Tₙ),
where Tₙ is the last parameter t and r⁽ⁿ⁾(Tₙ) is the last radius of the envelope of the nth catheter segment S⁽ⁿ⁾.

It can also be assumed advantageously that, after the first catheter segment is known, the following applies to the parameters of the center line of the (n+1)th catheter segment mentioned thereafter:

$$\begin{matrix}{a}_{11}^{( n + 1 )} = \frac{ⅆ}{ⅆ t} {x}^{( n )} ( t ) {\vert}_{{T}_{n}} = {a}_{12}^{( n )} {T}_{n} + {a}_{11}^{( n )} \\ {a}_{21}^{( n + 1 )} = \frac{ⅆ}{ⅆ t} {y}^{( n )} ( t ) {\vert}_{{T}_{n}} = {a}_{22}^{( n )} {T}_{n} + {a}_{21}^{( n )} \\ {a}_{31}^{( n + 1 )} = \frac{ⅆ}{ⅆ t} {z}^{( n )} ( t ) {\vert}_{{T}_{n}} = {a}_{32}^{( n )} {T}_{n} + {a}_{31}^{( n )}\end{matrix} \}$$

and/or that, after the first catheter segment is known, the following applies to the parameter of the radius of the envelope of the (n+1)th catheter segment mentioned thereafter:

$${b}_{1}^{( n + 1 )} = \frac{ⅆ}{ⅆ t} {r}^{( n )} ( t ) {\vert}_{{T}_{n}} = {b}_{2}^{( n )} {T}_{n} + {b}_{1}^{( n )} .$$

Furthermore, the virtual (n+1)th catheter segment S⁽ⁿ⁾ can be optimally adapted in a first step due to the fact that at the end point of the nth and, at the same time, the starting point of the (n+1)th center line, a spherical sector is described with a radius in the progressive direction. The spherical sector is used as “search space” for determining the optimum (n+1)th catheter segment. To adapt the catheter segment to the actual vessel, the catheter segment is selectively swung to and fro within the spherical segment (compare FIG. 3). For this purpose, crossover points of the center line through which the center line extends are determined on the spherical segment surface. In this context, a vessel branch can be recognized from the fact that more than two vessel crossovers are detected, wherein the virtual catheter can be calculated subsequently for each vessel branch.

A further advantageous embodiment of the method according to the invention provides that the following quality function E(aᵢⱼ⁽ⁿ⁾) is used as a measure of the optimum adaptation of the parameters aᵢⱼ⁽ⁿ⁾ of the virtual nth catheter segment S⁽ⁿ⁾ to the respective vessel section:
E(aᵢⱼ⁽ⁿ⁾)=\dot{E}_{Ex}+λE_{In},
where E_{Ex} is also called “external energy” and represents a measure of the quality of the adaptation of the virtual catheter segment to the vessel, E_{In} is called “internal energy” and represents a measure of the curvature of the virtual catheter segment and λ corresponds to a weighting factor between the “energies”. In this context, smaller “energy” values signify better adaptation to the real vessel.

The measure of the “external energy” E_{Ex} can be calculated, for example, by using the following formula:

$${E}_{Ex} = \frac{1}{\vert {V}_{n} \vert} {\sum }_{{\vec{r}}_{i} ∈ {V}_{n}} {f}_{{\vec{n}}_{i}} ( \vec{r} )$$

$$with$$

$${f}_{n} ( \vec{r} ) = \frac{\partial f}{\partial \vec{n}} ( \vec{r} ) = 〈 ∇ f ( \vec{r} ) , \vec{n} 〉 ,$$

where Vₙ is the volume of the nth catheter segment; n is the perpendicular from position rᵢ to the center line (normal vector) (compare FIG. 4); ∇f is the gradient of the function f.

The measure of the “internal energy” E_{In} can be calculated correspondingly by using the following formula:

$${E}_{In} = \frac{1}{{S}_{n}} {\int }_{t = 0}^{{T}_{n}} {κ}^{2} ( t ) {‖ {c}_{t}^{( n )} ( t ) ‖}_{{t}^{2}} ⅆ t$$

with the segment length Sₙ and the curvature of the center line

$$κ ( t ) = \frac{{‖ {c}_{t}^{( n )} ( t ) \times {c}_{tt}^{( n )} ( t ) ‖}_{{t}^{2}}}{{‖ {c}_{t}^{( n )} ( t ) ‖}_{{t}^{2}}^{3}} ,$$

where

$${c}_{t}^{( n )} = \frac{ⅆ}{ⅆ t} {c}^{( n )} ( t )$$

is the first derivation of c with respect to

$$t , {c}_{tt}^{( n )} = \frac{{ⅆ}^{2}}{ⅆ {t}^{2}} {c}^{( n )} ( t )$$

is the second derivation of c with respect to t and ∥ ∥ᵢ_{²} designates the Euclidean norm of a vector.

It is also advantageous that the calculation is continued until a threshold value for the quality of adaptation is reached or until a predetermined total length of the virtual catheter is reached or until a termination signal is received from the operator.

The context of at least one embodiment of the invention also includes a tomography system comprising a computing unit which has at least one stored computer program or program modules which, in one embodiment, wholly or partially executes/execute at least one embodiment of the method described above in operation on the computing unit of the tomography system.

Similarly, at least one embodiment of the invention also includes a storage medium which is integrated into a computing unit or is intended for a computing unit of a tomography system and at least one computer program or program modules is/are stored on it which, in one embodiment, at least partially executes/execute at least one embodiment of the method described above on the computing unit of the tomography system.

### BRIEF DESCRIPTION OF THE DRAWINGS

In the text which follows, at least one embodiment of the invention will be described in greater detail with reference to an example embodiment with the aid of the figures, wherein only the features necessary for understanding the invention are shown and the following reference symbols are used: 1: CT system; 2: first X-ray tube; 3: first detector; 4: second, optional X-ray tube; 5: second, optional detector; 6: gantry housing; 7: patient; 8: patient table; 9: system axis; 10: control and computing unit; 11: memory; 12: contrast medium pump; 13: ECG line; 14: control and data line; 15: control line of the contrast medium pump;

c⁽ⁿ⁾(t): center line of the nth segment with the run variable t; cₜ(t*): tangential vector; d: distance between voxel and center line; K: spherical segment; n: run variable of the catheter segments; \vec{n}: normal vector; Prg₁-Prgₙ: computer programs; Prgₓ: selected computer program; P: crossover point in the spherical segment; R: radius; r: voxel position; r⁽ⁿ⁾(t): radius of the nth segment with the run variable t; S⁽ⁿ⁾: nth catheter segment; t: continuing control variable; t*: parameter; u₁: longitudinal axis of the first catheter segment; x,y,z: Cartesian coordinates; θ: aperture angle.

In detail:

FIG. 1 shows the structure of the virtual catheter in the example of two successive catheter segments;

FIG. 2 shows a prototype of the virtual catheter in a screen display, start of the procedure;

FIG. 3 shows swinging of a catheter segment by predetermining crossover points on a spherical sector surface;

FIG. 4 shows determination of a voxel position as belonging to the catheter segment;

FIG. 5 shows a prototype of the virtual catheter as super-position in a screen display, 100% of the procedure carried out; and

FIG. 6 shows a CT system for carrying out the method according to an embodiment of the invention.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. Like numbers refer to like elements throughout. As used herein, the terms “and/or” and “at least one of” include any and all combinations of one or more of the associated listed items.

To determine the spread/extent of diseases of coronary vessels, conventional angiography of the coronary vessels is mostly used wherein a vessel catheter has to be placed for this purpose. This catheterization is an invasive imaging technique in which a catheter is introduced into an arm or a leg of the patient and is guided through the blood vessel system into the coronary vessels under guidance of two-dimensional imaging. At present, such heart catheterization is considered to be the standard method for visualizing blockages, narrowing or abnormalities in the coronary vessels.

The inventor, inspired by the conventional procedure of catheterization, proposes a novel technique for separating blood vessels from volume images by guiding a synthetic catheter model virtually through a three-dimensional image record generated by tomographic recordings of a patient. In the text which follows, an actual variant of an embodiment of the invention is described in detail without restriction of the general applicability of the invention.

An actual catheter model according to an embodiment of the invention includes a tubular generated surface with a center line in the interior. The center line c(t)=(x(t),y(t),z(t)) is described by three spatial coordinates x(t), y(t) and z(t) parameterized in t. The parameter t is a control variable which increases monotonously with the advance on the curve. The generated surface is arranged in such a manner that it has a circular, generally also ellipsoidal cross section. Its distance from the center line is described by the radius r(t) which, like the center line, also depends on the parameter t.

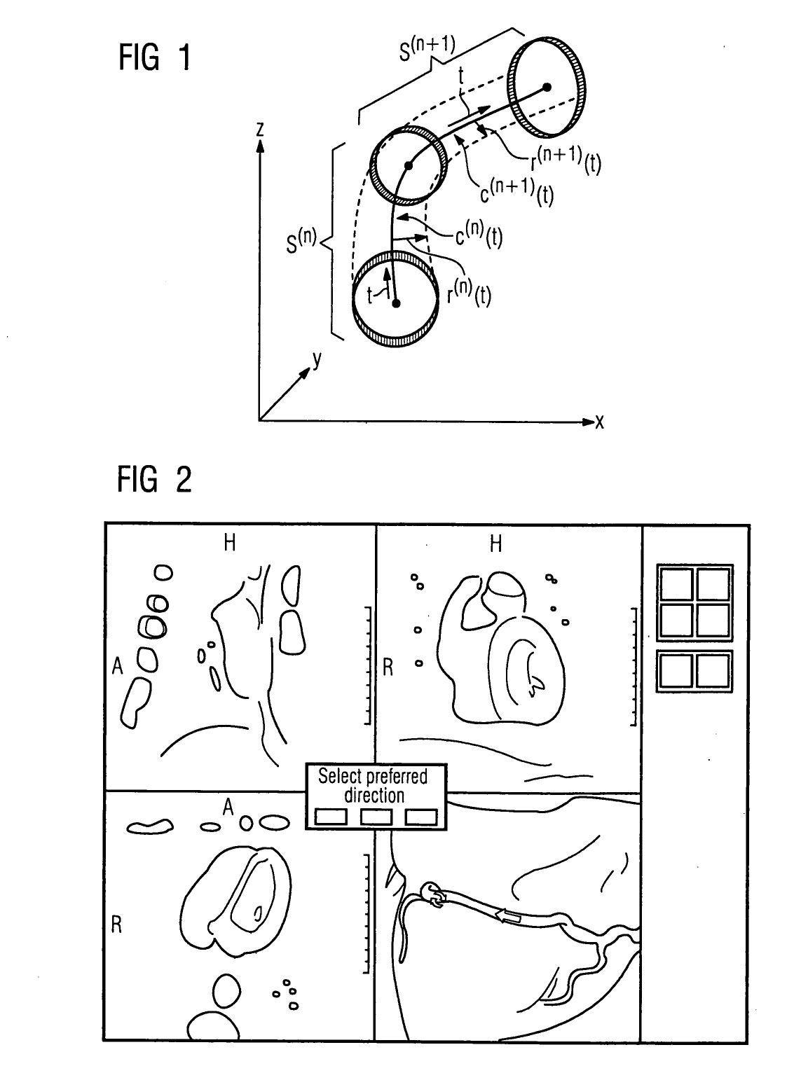

FIG. 1 shows such a catheter which includes short segments n and n+1 which are assembled in such a manner that the transitions of center line and radius are continuous and smooth at the joining points. That is to say, they should be formed ideally without discontinuities or, respectively, without sharp edges. This can be achieved by representing the components of center line and radius in quadratic splines, for example piecewise quadratic polynomials.

The successive catheter segments are numbered. The nth segment of the center line can then be described by the following formula:

$${\vec{c}}^{( n )} ( t ) = ( \begin{matrix}{x}^{( n )} ( t ) \\ {y}^{( n )} ( t ) \\ {z}^{( n )} ( t )\end{matrix} ) = ( \begin{matrix}\frac{1}{2} {a}_{12}^{( n )} {t}^{2} + {a}_{11}^{( n )} t + {a}_{10}^{( n )} \\ \frac{1}{2} {a}_{22}^{( n )} {t}^{2} + {a}_{21}^{( n )} t + {a}_{20}^{( n )} \\ \frac{1}{2} {a}_{32}^{( n )} {t}^{2} + {a}_{31}^{( n )} t + {a}_{30}^{( n )}\end{matrix} ) .$$

The coefficients a_{i,j}⁽ⁿ⁾, i=1, . . . , 3; j=0, . . . , 2 must be selected in such a manner that the curve segment is assembled flat with the preceding segment, that is to say without discontinuity and with the same direction of tangent, and that it joins the center of the underlying vessel piece as well as possible.

In the same manner, the radius of the nth catheter segment S⁽ⁿ⁾ is described by the formula

$${r}^{( n )} ( t ) = \frac{1}{2} {b}_{2}^{( n )} {t}^{2} + {b}_{1}^{( n )} t + {b}_{0}^{( n )} .$$

In this context, the coefficients bⱼ⁽ⁿ⁾, j=0, . . . , 2 are selected in such a manner that they approximate the genuine vessel surfaces and ensure smooth transitions between the successive surface segments.

Apart from the fact that this model has the appearance of genuine vessels—which normally do not have any discontinuities or sharp edges—the use of the quadratic splines has the advantage that many calculations for approximating the model to the records can be carried out analytically in mathematically closed form. This reduces the necessity of iterative estimations to a minimum and leads to precisely calculable and very time-efficient algorithms.

FIG. 2 shows a prototype of the virtual catheter and demonstrates its functionality in the segmentation of the coronary arteries from CTA data. In this arrangement, the first catheter segment is initiated by a single mouse click into the vessel and is segmented. This marking can be set in one of the standard 2-D views, e.g. sagital, coronal or axial, or alternatively in a 3-D volume display of the volume of interest. The initial direction of the first catheter segment can be determined as follows: starting from the marking, an adjacency-increasing algorithm starts, taking into consideration only voxels with a predetermined grayscale value—that is to say voxels from the vessel. This algorithm in each case looks for adjacent voxels which meet a particular criterion, for example a minimum HU value, and can thus be graded as belonging to the vessel.

After each expansion step of the algorithm, the axes of inertia u₁, u₂, u₃ and corresponding Eigen values λ₁≧λ₂≧λ₃ are calculated by determining the tensor of inertia of the current segment volume found. As soon as the segment volume exhibits a noticeable deflection along one of its axes of inertia, for example λ₁>>λ₂ and λ₁>>λ₃, the algorithm is stopped. The first catheter segment is then represented as a straight cylinder with the longitudinal axis u₁. The two smaller Eigen values 2 and 3 are used for calculating the initial radius. A small arrow in the recording on the bottom right in FIG. 2 shows the preferred direction of catheter propagation. The preferred direction of propagation can also be determined by the user, however. As an alternative, the catheter can also be started in both directions.

Once the nth catheter segment has been adapted to the data, the model automatically elongates itself by an (n+1)th segment. This (n+1)th segment is described by its 12 model parameters a_{i,j}⁽ⁿ⁺¹⁾ and bⱼ⁽ⁿ⁺¹⁾, i=1, . . . , 3; j=0, . . . , 2.

Eight of these parameters only have the purpose of meeting the smoothness criterion of the center line and the surface radius at the transition point between the individual segments. If it is assumed that the parameter t of the nth segment is in the interval [0, Tₙ], the steadiness condition of the center line and of the surface radius implies that the conditions

$$\begin{matrix}{a}_{10}^{( n + 1 )} = {x}^{( n )} ( {T}_{n} ) \\ {a}_{20}^{( n + 1 )} = {y}^{( n )} ( {T}_{n} ) \\ {a}_{30}^{( n + 1 )} = {z}^{( n )} ( {T}_{n} )\end{matrix} \} \\$$

apply to the steadiness of the center line and
b₀⁽ⁿ⁺¹⁾=r⁽ⁿ⁾(Tₙ)
applies to the steadiness of the surface radius.

From the smoothness conditions at the transition points, the following mathematical rules are obtained:

$$\begin{matrix}{a}_{11}^{( n + 1 )} = \frac{ⅆ}{ⅆ t} {x}^{( n )} ( t ) {\vert}_{{T}_{n}} = {a}_{12}^{( n )} {T}_{n} + {a}_{11}^{( n )} \\ {a}_{21}^{( n + 1 )} = \frac{ⅆ}{ⅆ t} {y}^{( n )} ( t ) {\vert}_{{T}_{n}} = {a}_{22}^{( n )} {T}_{n} + {a}_{21}^{( n )} \\ {a}_{31}^{( n + 1 )} = \frac{ⅆ}{ⅆ t} {z}^{( n )} ( t ) {\vert}_{{T}_{n}} = {a}_{32}^{( n )} {T}_{n} + {a}_{31}^{( n )}\end{matrix} \}$$

for the smoothness of the center line and

$${b}_{1}^{( n + 1 )} = \frac{ⅆ}{ⅆ t} {r}^{( n )} ( t ) {\vert}_{{T}_{n}} = {b}_{2}^{( n )} {T}_{n} + {b}_{1}^{( n )}$$

for the smoothness of the surface radius.

The four parameters a₁₂⁽ⁿ⁺¹⁾, a₂₂⁽ⁿ⁺¹⁾, a₃₂⁽ⁿ⁺¹⁾ and b₂⁽ⁿ⁺¹⁾ of the (n+1) model segment are thus the only free parameters of the next segment which are to be determined newly and the values of which are sought with respect to the best possible fit to the vessel considered.

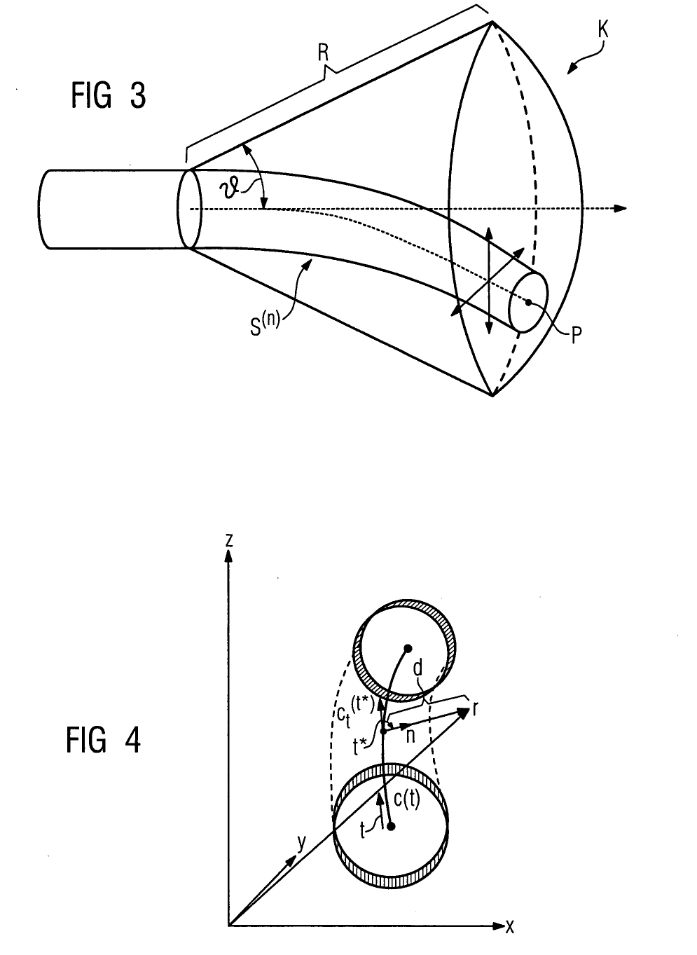

FIG. 3 shows how a given front segment S⁽ⁿ⁾ is swung/controlled in the area to be examined in order to find the optimum adaptation to the vessel. In the quality function, which is introduced in the next section, the quality of adaptation is represented. The area to be examined is determined in a solid angle element Ωε [symbol] ³ with predetermined aperture angle θ and fixed radius R. The center line of the catheter segment is swung about by predetermining points on the spherical sector surface K through which the elongation of the center line must go/penetrate. The extent by which the center line is bent is determined by varying the upper limit Tₙ of the parameter t. Low values of the parameter Tₙ result in a straight center line whereas higher values of Tₙ produce curves with greater curvature. For each given crossover point P and parameter Tₙ, the free parameters a₁₂⁽ⁿ⁾, a₂₂⁽ⁿ⁾ and a₃₂⁽ⁿ⁾ are determined in such a manner that the arc length Sₙ of the center line constantly maintains a predefined value. The relation between the arc length Sₙ and the associated maximum value of the parameter Tₙ of t is

$${S}_{n} ( {T}_{n} ) = {\int }_{t = 0}^{{T}_{n}} \sqrt{{( {x}_{t}^{( n )} ( t ) )}^{2} + {( {y}_{t}^{( n )} ( t ) )}^{2} + {( {z}_{t}^{( n )} ( t ) )}^{2}} ⅆ t .$$

If quadratic splines are used, the above integral can be solved analytically and allows an efficient and precise determination of the arc length. The inverse relation Tₙ(Sₙ) must be determined by an iterative numeric approximation as long as there is no analytic inversion of the above formula.

For each given center line configuration, the free model parameter b₂⁽ⁿ⁺¹⁾ must be varied for determining the associated optimum segment surface area. This process is iteratively repeated until the best center line and radius configuration for a given crossover point is found. The positions of the crossover points are then determined more closely iteratively in order to obtain the optimum segment model.

The center lines and radius adaptation are carried out for various segment lengths Sₙ. If the quality function described in the next section is normalized with respect to length, the values can be compared directly for selecting the optimum length of the current segment.

For a given set of model parameters a_{i,j}⁽ⁿ⁺¹⁾ and bⱼ⁽ⁿ⁺¹⁾, i=1, . . . , 3; j=0, . . . , 2, the quality of adaptation to the underlying image is determined by calculating a quality function. The quality function consists of two terms, an external energy and an internal energy. The external energy represents the quality of adaptation of the model to the image data and the internal energy describes the degree of curvature of the segment model.

The external energy is dependent on the fact that the average image intensity of a vessel cross section is typically maximum in the center and rapidly decreases with increasing distance from the center. The decrease in intensity is specified in units of the directional derivative of the image intensity f, which is perpendicular to the center line. With a given point r=(x,y,z) in space, the directional derivative of the intensity f in the direction n=(n₁, n₂, n₃) perpendicular to the center line is defined by

$${f}_{n} ( \vec{r} ) = \frac{\partial f}{\partial \vec{n}} ( \vec{r} ) = 〈 ∇ f ( \vec{r} ) , \vec{n} 〉 ,$$

where ∇f(\vec{r})=(fₓ(\vec{r}),f_{y}(\vec{r}),f_{z}(\vec{r}))^{T} designates the gradient of f at the point r. The model segment is optimum when the directional derivative

$${E}_{Ex} = \frac{1}{\vert {V}_{n} \vert} {\sum }_{{{r}^{->}}_{i} ∈ {V}_{n}} {f}_{{\vec{n}}_{i}} ( {\vec{r}}_{i} )$$

averaged over the volume Vₙ of the catheter segment is minimum.

The directional derivatives are negative when the intensity decreases. The sum runs over all voxels i, the positions rᵢ of which are within the given catheter segment. In contrast to existing approximations which are based on sending out search beams or calculated gradient information only from the vessel surface, the proposed energy term evaluates the information of all voxels within the given catheter segment. The proposed and preferred approximation is thus less sensitive in the case of noise or vessel surfaces in vessels which adjoin other high-contrast objects such as ventricles and bones.

The external energy depends on the center line and on the radius of the tubular surface area and takes into consideration their mutual dependence. The vessel intensity normally decreases rapidly in the vertical direction from the center line, the energy becomes minimum when the center line of the model is aligned on the vessel. The radius is similarly forced to adapt itself to the true vessel thickness. If the radius is greater than the present vessel cross section, the order of magnitude of the direct derivative is small in the periphery. If the sum of the directional derivative is divided by the higher volume, E_{Ex} decreases. If the intensity is at a maximum in the center of the cross section, the first derivative in the direction of the normal approaches zero the closer one comes to the center. Thus, too small a surface radius also leads to a smaller energy E_{Ex}.

The decisive problem in calculating the above formula is the determination of the amount of voxels located within the present model segment and the calculation of the corresponding normal to the center line. As shown in FIG. 4, the normal for a voxel in the spatial element Ω is calculated by dropping the perpendicular on the center line. For a given voxel position rᵢ, the parameter t* is selected in such a manner that the tangential vector cₜ(t*) is perpendicular to the connecting vector between the center line position c(t*) and the voxel rᵢ, for example
<\vec{c}ₜ(t*),\vec{r}ᵢ−\vec{c}(t*)>=0,
where the left-hand expression of this equation is the inner product of two vectors.

To solve the above equation with respect to t*, the root of a third-order polynomial must be determined in t which can be calculated analytically in a mathematically closed form, that is to say by directly solving an equation. Depending on the situation, the cubic polynomial either has one real and two complex zero positions or three real zero positions. The parameter t* should be the smallest real zero position located in the interval [0, Tₙ]. The distance dᵢ of voxel rᵢ from the center line is given by the following formula
dᵢ=∥\vec{r}ᵢ−\vec{c}(t*)∥ₗ_{²}.

The normal vector \vec{n}ᵢ is then defined as

$${\vec{n}}_{i} = \frac{{\vec{r}}_{i} - \vec{c} ( {t}^{*} )}{{ⅆ}_{i}} .$$

The voxel position rᵢ is within the catheter segment when dᵢ≦r(t*).

The inner energy is dependent on the total curvature of the center line of the model. The local curvature κ(t) of the curve c⁽ⁿ⁾(t) is defined as

$$κ ( t ) = \frac{{‖ {c}_{t}^{( n )} ( t ) ⨯ {c}_{tt}^{( n )} ( t ) ‖}_{{l}^{2}}}{{‖ {c}_{t}^{( n )} ( t ) ‖}_{{l}^{2}}^{3}} ,$$

where “x” is the cross product between two vectors. The path integral of the squared curvature along the curve defines the so-called energy of the bend. For a straight line, the energy of the bend is zero and the more frequently and greater a curve is bent, the greater it becomes. The inner energy of the catheter segment is here the bowing energy normalized to the arc length of the curve Sₙ, for example

$${E}_{In} = \frac{1}{{S}_{n}} {\int }_{t = 0}^{{T}_{n}} {κ}^{2} ( t ) {‖ {c}_{t}^{( n )} ( t ) ‖}_{{l}^{2}} ⅆ t .$$

If quadratic splines are used, the integral can be solved analytically with a closed solvable equation. The inner energy induces a certain stiffness in the catheter model. This prevents extreme curvatures of the model in the regions where the signal-noise ratio of the external energy is low.

The catheter segment is considered to be optimum when the total energy
E(a₁₂⁽ⁿ⁾,a₂⁽ⁿ⁾,a₃₂⁽ⁿ⁾,b₂⁽ⁿ⁾)=E_{Ex}+λE_{In}
is minimum, wherein λ designates a constant weighting factor which describes the relative influence of the external energy in the total energy. Minimization of the quality function can be interpreted in such a manner that a catheter segment with a rather smooth center line is obtained which fits the underlying vessel data as well as possible. Since the internal and external energy is normalized to the volume and the length of the segment, catheter segments with different volumes and lengths can also be compared.

According to at least one embodiment of the invention, the iterative process of segment adaptation and catheter elongation runs until one of the following events occurs:
1. The catheter reaches its predefined length.
2. The quality function becomes too small compared with the preceding elements (reaching the vessel end or the catheter leaves the correct path).
3. Premature termination of the adaptation process by the operator.

if necessary, the operator can modify the last catheter segment—during the process of virtual catheter construction—by drawing with the mouse at the catheter end and can continue the process if desired.

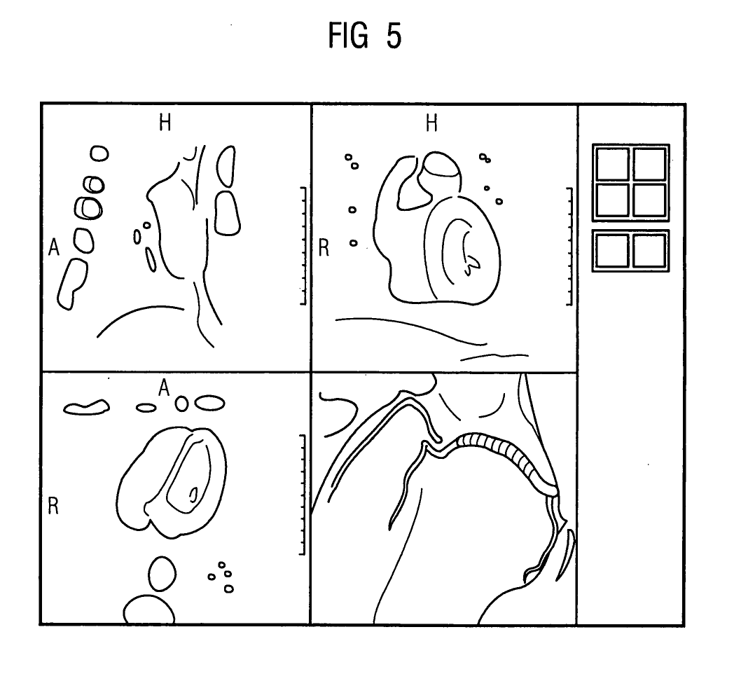

The capability of the virtual catheter to subdivide coronary arteries into individual segments from volume data of a CTA (computer tomography angiography) is shown in FIG. 5. The result of the segmentation is displayed both as an overlay over volume data rendered and via a two-dimensional sector image from an MPR (multiplanar reconstruction) recording. The overlay can be updated as the catheter proceeds. Useful model parameters, for example the local surface and the radius, can be displayed at the desired point by a mouse click.

In addition, the operator can also select a subregion of the segmented vessel by two mouse clicks on the starting and end points. Both points can slide interactively along the center line for reaching a particular segment of interest. Similarly, the parameters of volume, bowing energy, average of minimum and maximum radius and average of minimum and maximum HU values can be displayed for the selected vessel section. Furthermore, other clinically relevant parameters can also be derived from the model.

The proposed method according to an embodiment of the invention utilizes the tubular vessel geometry for achieving efficient and rugged segmentation. Clinically relevant parameters such as vessel radius, volume, surface, etc., are given by the adapted model and do not need to be extracted from binary masks. Thus, segmentation of a vessel section of interest can be obtained with a single mouse click. The operator can predefine a particular length of the vessel which is to be segmented. If the catheter is visualized during the calculation, the operator can abort or modify the process, if desired.

The technical/algorithmic progress can be summarized as follows:
The term of external energy of the quality function describes the total data image included in a model and leads to an increasing ruggedness with regard to the signal-noise ratio.
The vessel center line and the surface area are adapted in a single process which makes use of their mutual dependence.
The use of second-order splines has a number of advantages:
   Many model parameters (volume, arc length, etc.) can be calculated efficiently and precisely if closed equations are used.
   The model enables an uninterrupted and smooth center line and surface area to be represented.

As a precaution, it should be noted that the method according to an embodiment of the invention is not restricted to catheter segments having a round cross section but can also be extended to elliptical cross sections in a generalization of the description listed above. By accommodating further parameters, an even better adaptation of the catheter segments to the course and external form of the vessels considered is thus possible, the calculation in closed form still being analytically possible.

It must also be pointed out that the method according to an embodiment of the invention can be carried out not only in conjunction with CT data but also with tomographic MR data. In this context, it is not HU values but other grayscale values or color values of the image representation which are used as criterion of the adaptation.

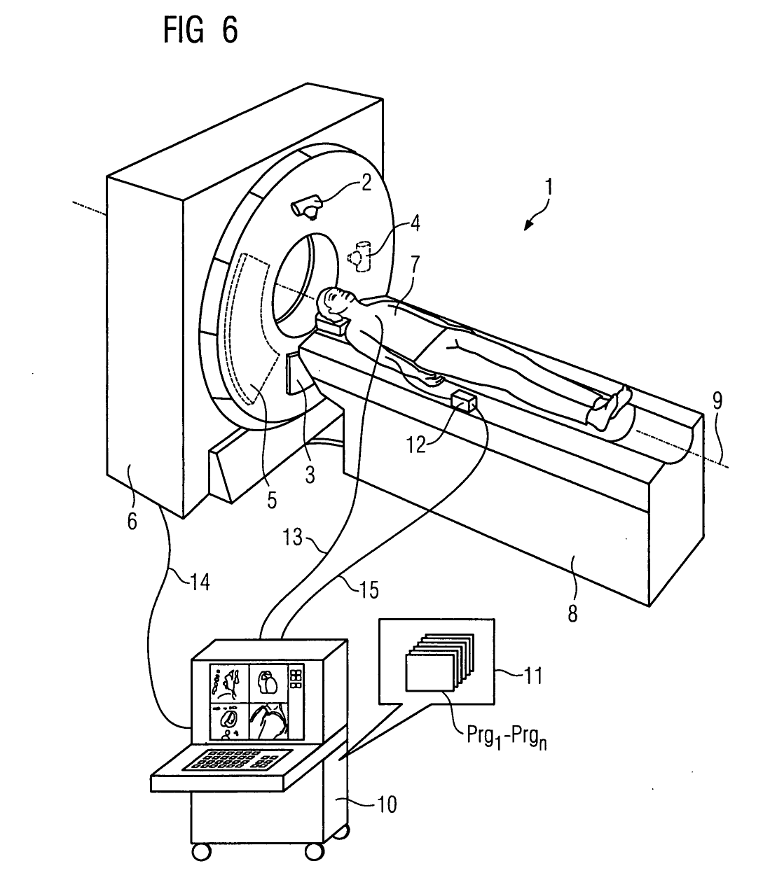

FIG. 6, finally, shows a CT system 1 according to an embodiment of the invention by way of example, with a gantry housing 6 in which the rotatable gantry frame is located which, however, is not explicitly drawn here. On the patient table 8, a patient 7 to be examined is lying who, during the rotation of the gantry frame, can be pushed along the system axis 9 through an opening in the gantry housing which approximately describes the scanning area of the focus/detector systems. As an example, such a CT system has at least one focus/detector system including an X-ray tube 2 with the focus generated there and an oppositely located detector system 3 with at least one detector row, in most cases a multiplicity of detector rows arranged next to one another. To improve the recording performance and/or temporal resolution, one or two further focus/detector systems can be optionally installed. In the present representation, an optional second focus/detector system with a second X-ray tube 4 and a second detector 5 are indicated dashed.

The CT system 1 is controlled by a control and computing unit 10 via the control and data line 14 wherein the reconstruction, evaluation and segmentation according to the invention of the data measured in the detectors can also be effected on this computing unit with the aid of the programs Prg₁ to Prgₙ stored in an internal memory 11 or on a storage medium. In addition, an optionally usable contrast medium pump 12 with its control line 15 is shown in order to provide for the contrast medium application normally used in cardio or general vessel recordings. Control is effected via the control and computing unit 10. In addition, an ECG line 13 from the patient 7 to the control and computing unit 10 is shown which also handles the operation of an ECG as is necessary in most cases with cardio recordings, for example.

It is additionally also pointed out that within the context of an embodiment of the invention, any type of generation of the recordings, that is to say with or without contrast medium and/or with or without ECG triggering and/or with one or more focus/detector systems, is intended to be contained.

However, the processing of the volume data and/or the performance of the method according to an embodiment of the invention can also be transferred to other computing stations without departing from the context of the invention. It is also understood that the aforementioned features of embodiments of the invention can be used not only in the combination specified in each case but also in other combinations or by themselves without departing from the context of the invention.

Overall, at least one embodiment of the invention thus proposes a method for mathematically generating a virtual catheter in tomographic volume data in which the virtual catheter is assembled from a multiplicity of catheter segments, each of which can be separately described in a closed mathematical expression and which are in each case approximated to the measured volume data by error calculation and variation of their parameters. In particular, transition criteria between the surfaces and the center lines of the virtual segments which create smooth transitions and thus already predetermine a multiplicity of parameters of the segment following in each case are used in order to reduce the parameters to be adapted. The description of the envelope of the segments and of the center line of the segments by means of second-degree polynomials is particularly suitable for representation in a mathematically closed form.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks™, cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for vessel segmentation in tomographic volume data, comprising:
reconstructing tomographic volume data of a patient, vessel representations including image values which significantly differ from the environment;
receiving a picture element in the volume examined (voxel), marked by an operator in a tomographic representation, as starting point in a vessel of interest;
automatically marking adjacent voxels around this starting point, the image values of which are in the vicinity of the originally marked voxel within predetermined image value limits until the sum of all marked voxels reveals a preferred spatial orientation and a first radius perpendicularly to the preferred spatial orientation;
joining a multiplicity of virtual catheter segments, beginning at least in one direction of the preferred spatial orientation, which have a center line and at least one of a circular envelope with one radius and an ellipsoidal envelope with two semi-axes and which meet the following conditions:
each component of the center line of a catheter segment is described by a second-degree polynomial,
the radius of the envelope of each catheter segment is described by a second-degree polynomial,
the center lines of adjacent catheter segments merge into one another, and
the envelopes of adjacent catheter segments merge into one another, wherein for each continuing catheter segment, the parameters of the describing polynomials are varied until the course of the virtual catheter is optimally adapted to the course of the vessel.

2. The method as claimed in claim 1, wherein the continuing catheter segments continuously merge into one another at least with respect to their center lines.

3. The method as claimed in claim 2, wherein the center lines of the continuing catheter segments exhibit the same spatial direction at their contact points.

4. The method as claimed in claim 1, wherein the envelopes of adjacent catheter segments continuously merge into one another.

5. The method as claimed in claim 4, wherein the surface tangents of the envelopes of adjacent catheter segments continuously merge into one another.

6. The method as claimed in claim 1, wherein the center lines (c⁽ⁿ⁾(t)) of the catheter segments (S⁽ⁿ⁾) are described by the following formula in the Cartesian system of coordinates:
$${\vec{c}}^{( n )} ( t ) = ( \begin{matrix}{x}^{( n )} ( t ) \\ {y}^{( n )} ( t ) \\ {z}^{( n )} ( t )\end{matrix} ) = ( \begin{matrix}\frac{1}{2} {a}_{12}^{( n )} {t}^{2} + {a}_{11}^{( n )} t + {a}_{10}^{( n )} \\ \frac{1}{2} {a}_{22}^{( n )} {t}^{2} + {a}_{21}^{( n )} t + {a}_{20}^{( n )} \\ \frac{1}{2} {a}_{32}^{( n )} {t}^{2} + {a}_{31}^{( n )} t + {a}_{30}^{( n )}\end{matrix} ) ,$$
where n is the index for the consecutive number of the catheter segment, x, y, z are the Cartesian coordinates, t is an arbitrary parameter which increases monotonously with the length of the center line and aᵢⱼ are the parameters to be determined in the polynomials.

7. The method as claimed in claim 1, wherein the radius of the center line of the catheter segments is described by the following formula in the Cartesian system of coordinates:
$${r}^{( n )} ( t ) = \frac{1}{2} {b}_{2}^{( n )} {t}^{2} + {b}_{1}^{( n )} t + {b}_{0}^{( n )} ,$$
where n is the consecutive number of the catheter segment, x, y, z are the Cartesian coordinates, t is an arbitrary parameter which increases monotonously with the length of the center line and bᵢ are the parameters to be determined in the polynomials.

8. The method as claimed in claim 6, wherein, after the nth catheter segment (S⁽ⁿ⁾) is known, the following applies to the parameters of the center line of the (n+1)th catheter segment mentioned thereafter:
$$\begin{matrix}{a}_{10}^{( n + 1 )} = {x}^{( n )} ( {T}_{n} ) \\ {a}_{20}^{( n + 1 )} = {y}^{( n )} ( {T}_{n} ) \\ {a}_{30}^{( n + 1 )} = {z}^{( n )} ( {T}_{n} )\end{matrix} \} ,$$
where Tₙ is the last parameter t and x⁽ⁿ⁾(Tₙ) y⁽ⁿ⁾(Tₙ) z⁽ⁿ⁾(Tₙ) are the last coordinates of the center line of the nth catheter segment.

9. The method as claimed in claim 7, wherein, after the nth catheter segment (S⁽ⁿ⁾) is known, the following applies to the parameters of the radius of the envelope of the (n+1)th catheter segment (S⁽ⁿ⁺¹⁾) mentioned thereafter:
b₀⁽ⁿ⁺¹⁾=r⁽ⁿ⁾(Tₙ),
where Tₙ is the last parameter t and r⁽ⁿ⁾(Tₙ) is the last radius of the envelope of the nth catheter segment S⁽ⁿ⁾.

10. The method as claimed in claim 6, wherein, after the nth catheter segment (S⁽ⁿ⁾) is known, the following applies to the parameters of the center line of the (n+1)th catheter segment mentioned thereafter:
$$\begin{matrix}{a}_{11}^{( n + 1 )} = \frac{ⅆ}{ⅆ t} {x}^{( n )} ( t ) {\vert}_{{T}_{n}} = {a}_{12}^{( n )} {T}_{n} + {a}_{11}^{( n )} \\ {a}_{21}^{( n + 1 )} = \frac{ⅆ}{ⅆ t} {y}^{( n )} ( t ) {\vert}_{{T}_{n}} = {a}_{22}^{( n )} {T}_{n} + {a}_{21}^{( n )} \\ {a}_{31}^{( n + 1 )} = \frac{ⅆ}{ⅆ t} {z}^{( n )} ( t ) {\vert}_{{T}_{n}} = {a}_{32}^{( n )} {T}_{n} + {a}_{31}^{( n )}\end{matrix} \} .$$

11. The method as claimed in claim 7, wherein, after the first catheter segment (S⁽ⁿ⁾) is known, the following applies to the parameter of the radius of the envelope of the (n+1)th catheter segment mentioned thereafter:
$${b}_{1}^{( n + 1 )} = \frac{ⅆ}{ⅆ t} {r}^{( n )} ( t ) {\vert}_{{T}_{n}} = {b}_{2}^{( n )} {T}_{n} + {b}_{1}^{( n )} .$$

12. The method as claimed in claim 1, wherein the virtual (n+1)th catheter segment is adapted in a first step due to the fact that at the end point of the nth and, at the same time, the starting point of the (n+1)th center line, a spherical segment is described with a radius in the progressive direction, for adapting the catheter segment to the actual vessel, the catheter segment is selectively swung to and fro within the spherical segment and on the spherical segment surface, crossover points of the center line through which the center line extends are determined.

13. The method as claimed in claim 12, wherein a vessel branch is recognized from the fact that a quality function assumes a distinct minimum at more than one point on the spherical segment surface.

14. The method as claimed in claim 12, wherein the virtual catheter is calculated for each vessel branch.

15. The method as claimed in claim 1, wherein the following quality function E(aᵢⱼ⁽ⁿ⁾) is used as a measure of the optimum adaptation of the parameters aᵢⱼ⁽ⁿ⁾ of the virtual nth catheter segment (S⁽ⁿ⁾) to the respective vessel section:
E(aᵢⱼ⁽ⁿ⁾)=E_{Ex}+λE_{In},
where E_{Ex} (“external energy”) represents a measure of the quality of the adaptation of the virtual catheter segment to the vessel, E_{In} (“internal energy”) represents a measure of the curvature of the virtual catheter segment and λ represents a weighting factor, and smaller values correspond to better adaptation.

16. The method as claimed in claim 15, wherein the measure of the “external energy” E_{Ex} is calculated by using the following formula:
$${E}_{Ex} = \frac{1}{\vert {V}_{n} \vert} {\sum }_{{\vec{r}}_{i} ∈ {V}_{n}} {f}_{{\vec{n}}_{i}} ( {\vec{r}}_{i} )$$
$$with$$
$${f}_{n} ( \vec{r} ) = \frac{\partial f}{\partial \vec{n}} ( \vec{r} ) = 〈 ∇ f ( \vec{r} ) , \vec{n} 〉 ,$$
where Vₙ is the volume of the nth catheter segment; n is the perpendicular from position rᵢ to the center line (normal vector); ∇f is the gradient of the function f.

17. The method as claimed in claim 14, wherein the measure of the “internal energy” E_{In} is calculated by using the following formula:
$${E}_{In} = \frac{1}{{S}_{n}} {\int }_{t = 0}^{{T}_{n}} {K}^{2} ( t ) {‖ {c}_{t}^{( n )} ( t ) ‖}_{{l}^{2}} ⅆ t , \\ with$$
$$K ( t ) = \frac{{‖ {c}_{t}^{( n )} ( t ) ⨯ {c}_{u}^{( n )} ( t ) ‖}_{{l}^{2}}}{{‖ {c}_{t}^{( n )} ( t ) ‖}_{{l}^{2}}^{3}}$$
the curvature of the center line and Sₙ the segment length.

18. The method as claimed in claim 1, wherein the calculation of the catheter segments is continued until a threshold value for the quality of adaptation is reached.

19. The method as claimed in claim 1, wherein the calculation of the catheter segments is continued until a predetermined total length of the virtual catheter is reached.

20. The method as claimed in claim 1, wherein the calculation of the catheter segments is continued until a termination signal is received from the operator.

21. A tomography system, comprising:
a computing unit to store at least one computer program or program module which, when executed on the computing unit, causes the computing unit to perform at least the following:
reconstructing tomographic volume data of a patient, vessel representations including image values which significantly differ from the environment;
receiving a picture element in the volume examined (voxel), marked by an operator in a tomographic representation, as starting point in a vessel of interest;
automatically marking adjacent voxels around this starting point, the image values of which are in the vicinity of the originally marked voxel within predetermined image value limits until the sum of all marked voxels reveals a preferred spatial orientation and a first radius perpendicularly to the preferred spatial orientation;
joining a multiplicity of virtual catheter segments, beginning at least in one direction of the preferred spatial orientation, which have a center line and at least one of a circular envelope with one radius and an ellipsoidal envelope with two semi-axes and which meet the following conditions:
each component of the center line of a catheter segment is described by a second-degree polynomial,
the radius of the envelope of each catheter segment is described by a second-degree polynomial,
the center lines of adjacent catheter segments merge into one another, and
the envelopes of adjacent catheter segments merge into one another, wherein for each continuing catheter segment, the parameters of the describing polynomials are varied until the course of the virtual catheter is optimally adapted to the course of the vessel.

22. A storage medium, at least one of integrated into a computing unit and for a computing unit of a tomography system, storing at least one computer program or program module which, when executed, causes the computing unit to perform the method as claimed in claim 1.

23. The method as claimed in claim 7, wherein, after the nth catheter segment (S⁽ⁿ⁾) is known, the following applies to the parameters of the center line of the (n+1)th catheter segment mentioned thereafter:
$$\begin{matrix}{a}_{10}^{( n + 1 )} = {x}^{( n )} ( {T}_{n} ) \\ {a}_{20}^{( n + 1 )} = {y}^{( n )} ( {T}_{n} ) \\ {a}_{30}^{( n + 1 )} = {z}^{( n )} ( {T}_{n} )\end{matrix} \} ,$$
where Tₙ is the last parameter t and x₍ₙ₎(Tₙ), y⁽ⁿ⁾(Tₙ), z⁽ⁿ⁾(Tₙ) are the last coordinates of the center line of the nth catheter segment.

24. The method as claimed in claim 8, wherein, after the nth catheter segment (S⁽ⁿ⁾) is known, the following applies to the parameters of the radius of the envelope of the (n+1)th catheter segment (S⁽ⁿ⁺¹⁾) mentioned thereafter:
b₀⁽ⁿ⁺¹⁾=r⁽ⁿ⁾(Tₙ),
where Tₙ is the last parameter t and r⁽ⁿ⁾(Tₙ) is the last radius of the envelope of the nth catheter segment S⁽ⁿ⁾.

25. The method as claimed in claim 23, wherein, after the nth catheter segment (S⁽ⁿ⁾) is known, the following applies to the parameters of the radius of the envelope of the (n+1)th catheter segment (S⁽ⁿ⁺¹⁾) mentioned thereafter:
b⁽ⁿ⁺¹⁾=r⁽ⁿ⁾(Tₙ)
where Tₙ is the last parameter t and r⁽ⁿ⁾(Tₙ) is the last radius of the envelope of the nth catheter segment S⁽ⁿ⁾.

26. The method as claimed in claim 15, wherein the measure of the “internal energy” E_{In} is calculated by using the following formula:
$${E}_{In} = \frac{1}{{S}_{n}} {\int }_{t = 0}^{{T}_{n}} {K}^{2} ( t ) {‖ {c}_{t}^{( n )} ( t ) ‖}_{{l}^{2}} ⅆ t , \\ with$$
$$K ( t ) = \frac{{‖ {c}_{t}^{( n )} ( t ) ⨯ {c}_{u}^{( n )} ( t ) ‖}_{{l}^{2}}}{{‖ {c}_{t}^{( n )} ( t ) ‖}_{{l}^{2}}^{3}}$$
the curvature of the center line and Sₙ the segment length.
